(19) United States
(12) Patent Application Publication
(10) Pub. No.: US 2025/0241946 A1
(43) Pub. Date: Jul. 31, 2025

(54) MULTIVITAMINS FORMULATED TO DECREASE RISK FOR URINARY STONE FORMATION

(71) Applicant: Alaramed GM, LLC, Loma Linda, CA (US)

(72) Inventors: D. Duane BALDWIN, Loma Linda, CA (US); Antonio N. RUSCITTO, Franklin, TN (US)

(73) Assignee: Alaramed GM, LLC, Loma Linda, CA (US)

(21) Appl. No.: 19/041,701

(22) Filed: Jan. 30, 2025

Related U.S. Application Data
(60) Provisional application No. 63/627,011, filed on Jan. 30, 2024.

(51) Int. Cl. A61K 33/06; A61K 9/00; A61K 31/375; A61K 31/593; A61K 31/6615; A61K 36/88

(52) U.S. Cl. CPC A61K 33/06; A61K 9/0056; A61K 31/375; A61K 31/593; A61K 31/6615; A61K 36/88

(57) ABSTRACT

The present invention relates to a multivitamin formulation for reducing the risk of kidney stone formation. Such formulations comprise a reduction or elimination in one or more of one or more of vitamin C, vitamin D3, and/or calcium. The multivitamin formulations of the present invention may further include magnesium citrate, inositol hexaphosphate (IP-6), and/or banana stem extract. Also provided is a method for customizing a multivitamin formulation for reducing the risk of kidney stone formation in a patient. The method comprises assessing one or more criteria selected from history and physical exam, serum studies, spot urine parameters or 24 hour urine collections. Based on this assessment, a custom multivitamin formulation can be administered in accordance with the formulations described herein.

### CROSS-REFERENCE TO RELATED APPLICATION

This application claims the benefit of U.S. Prov. App. Nos. 63/627,011 (filed Jan. 30, 2024), which is incorporated by reference herein in its entirety.

### BACKGROUND

Vitamin C supplements are some of the most common dietary supplements consumed by the general population, with a widespread belief of boosting immune function and preventing common colds. More recently with the COVID-19 pandemic, drastic increases in the sale of vitamin C supplements were noted globally, and various demographic studies reported vitamin C to be the most commonly consumed supplement during the pandemic.

Commonly reported adverse effects of vitamin C supplements include diarrhea and gastrointestinal upset, particularly with larger doses. However, one important yet seldom discussed side effect of Vitamin C is the risk for urolithiasis. Consequently, physicians have reported an increase in the incidence and severity of kidney stone disease during the pandemic, attributed to increased supplement consumption.

Vitamin C, or ascorbic acid, is an essential micronutrient that plays an integral role in both innate and adaptive immune functions. It is required as a cofactor in several key enzymatic processes and is a potent antioxidant that can prevent cell damage from daily oxidative stress. The benefit of vitamin C in preventing the common cold originates from research conducted in the 1940s-1970s, and was popularized by Linus Pauling in 1970. Since then, vitamin C supplements, either alone or as part of multivitamin preparations, are widely available and consumed by the general population.

Despite this older evidence and popular belief, several studies have been published with contradicting and conflicting evidence. In 2013, a large Cochrane systematic review and meta-analysis including studies on the incidence of the common cold among the general population taking vitamin C supplements compared to placebo, reported a pooled relative risk of 0.97, indicating no clinical benefit of vitamin C in reducing the incidence of the common cold. Regarding the duration of the common cold, an 8-14% reduction was reported by the previous review, as well as a reduction in severity with vitamin C supplementation. However, they recognized a major limitation of variability in defining common cold severity across the included studies.

Similar to its effects on the common cold, evidence regarding the benefit of vitamin C on COVID is also variable. Some cross-sectional studies have demonstrated improved immune status and lower odds of severe COVID with vitamin C supplements, while two randomized controlled trials demonstrated no benefit of vitamin C in reducing COVID symptoms or duration in the outpatient setting. Nevertheless, vitamin C was the most common supplement utilized during COVID, leading to more and worse kidney stone formation. This was further compounded by the COVID restrictions and delays, resulting in even more complicated presentations and management of urolithiasis during the pandemic. Consequently, the increased consumption of vitamin C supplements may substantially contribute to the burden of managing urologic conditions globally without conferring actual benefits in protecting against COVID symptoms or severity.

The risk of urolithiasis with vitamin C intake was investigated by Ferraro et al in a longitudinal cohort study. In their study, 156,735 women and 40,536 men were included with a median follow-up of 11.3-11.7 years and were asked to complete food frequency questionnaires every 4 years that assessed their dietary and supplemental intake of vitamin C. During the follow-up period, 6,245 new urolithiasis cases were recorded. On adjusted analysis, total vitamin C intake was found to be significantly associated with the incidence of stones in men (HR=1.43, p=0.005), but not in women (HR=0.99). When analyzing dietary and supplemental vitamin C separately, there was no association between dietary vitamin C and stones in any cohort, however supplemental vitamin C was significantly associated with stone formation in men. Similarly, a cohort of 23,355 men from Sweden were followed for 11 years and use of vitamin C supplements was significantly associated with a 2X increased risk of kidney stones.

The main mechanism attributed to stone formation with vitamin C supplements is the increased oxalate that results from breakdown of ascorbic acid. This increased oxalate is eliminated in urine and can therefore precipitate with calcium and form calcium oxalate stones. In a recently performed and not yet published meta-analysis we evaluated the risk of vitamin C supplementation upon 24 hour urine parameters to determine the risk of stone formation. Our meta-analysis confirmed these findings reporting a significant increase in urinary oxalate excretion by approximately 10 mg/24 h after oral vitamin C supplementation. This was consistent for both SF and healthy volunteers who had no prior urolithiasis, highlighting a potential risk that anyone who consumes vitamin C supplements may be at a higher risk of kidney stones. In line with this, a significantly higher calcium oxalate supersaturation with vitamin C compared to placebo was reported for both SF and NSF.

Although numerous studies have been published on urinary oxalate after vitamin C and have reported conflicting results, many older investigations were criticized for the method used to measure urinary oxalate, as vitamin C excreted in urine can be converted ex vivo into oxalate. In our review, we used stringent inclusion criteria and only allowed studies with validated methods for urinary oxalate measurement or those that used stabilizing agents to prevent this ex vivo conversion. Furthermore, measurement of 24-h urinary creatinine was a requirement to ensure appropriate interpretation of 24-h urine parameters by the included studies.

Recent studies have shown no significant effect of vitamin C on urinary citrate, consistent with prior studies that showed no significant change in urinary citrate with vitamin C supplementation. Maintaining adequate urinary citrate levels is important in stone prevention. Citrate is known to be protective against calcium stones, as it forms a soluble complex in urine and prevents aggregation and growth of crystals. A large cross-sectional study on 2,561 individuals reported a significant decrease in urinary citrate among men who consume vitamin C but not women. This decrease in urinary citrate was hypothesized to be due to competitive inhibition of citrate absorption in the gut by vitamin C, as well as increased citrate reabsorption in the kidneys due to vitamin C-related urinary acidification.

The recommended daily intake of vitamin C for adults is 90 mg for males and 75 mg for females, and a variety of fruit and vegetable sources can provide more than adequate levels. For example, an orange contains 53.2 mg, and a red pepper contains 127.7 mg. Various studies have investigated the effects of natural vitamin C sources on urinary parameters. In one report, orange juice led to significantly higher urinary citrate compared to baseline with no significant increase in urinary oxalate. Similarly, consumption of diet lemonade was found to significantly increase urinary citrate and reduce calcium oxalate supersaturation, with no effect on oxalate excretion. A meta-analysis including 8 studies on citrus-based products reported significantly higher urinary citrate compared to controls (MD=125.5; p<0.001) and no difference in urine volume, calcium, or oxalate levels. Accordingly, rather than relying on supplements, consumption of vitamin C should be encouraged from natural sources that can provide the necessary intake required for proper immune function and without the added risk of urolithiasis. In fact, these sources may help prevent stone formation in some patients by increasing urinary citrate excretion.

What is still needed is a customizable multivitamin formulation that decreases the risk for formation of the most common type of kidney stones.

### SUMMARY OF THE INVENTION

The present invention relates to a multivitamin formulation for reducing the risk of kidney stone formation, preferably comprising less than about 100 mg vitamin C, more preferably less than 20 mg vitamin C, and most preferably substantially no vitamin C. The present invention also relates to a multivitamin formulation for reducing the risk of kidney stone formation, preferably comprising less than about 25 mcg vitamin D3, more preferably less than about 5 mcg vitamin D3, and most preferably substantially no vitamin D3. The present invention also relates to a multivitamin formulation for reducing the risk of kidney stone formation, preferably comprising less than about 250 mg calcium, more preferably less than about 25 mg calcium, and most preferably substantially no calcium. The multivitamin formulation of the present invention may also include the addition of magnesium citrate, inositol hexaphosphate (IP-6), and/or banana stem extract. The multivitamin formulation of the present invention may be administered orally in one or more tablets, capsules, gummies, powdered packet formulation, liquid formulation, or as a drink.

Also provided is a method for customizing a multivitamin for reducing the risk of kidney stone formation in a patient. The method comprises assessing one or more criteria selected from history and physical exam, serum studies, spot urine parameters or 24 hour urine collections. Based on this assessment, a custom multivitamin can be administered having a formulation in accordance with certain teachings of the present invention.

### DETAILED DESCRIPTION

The present invention relates to a multivitamin that contains no Vitamin C or reduced amounts of Vitamin C, thereby reducing the risk for formation of the most common type of kidney stone (calcium oxalate). The present invention also relates to a multivitamin that contains very low or no amounts of Vitamin D3, thereby reducing the risk for formation of calcium-based stones. The present invention also relates to a multivitamin that contains very low or no amounts of calcium, thereby reducing the risk for formation of calcium-based stones. The present invention also relates to a method for customizing multivitamins based upon dietary consumption, fluid intake, spot urine parameters or 24 hour urine parameters from standardized or proprietary analyses, to reduce the risk of kidney stone formation. The present invention also relates to a method for customizing multivitamins based upon metabolic conditions including Gout, diabetes, parathyroid hormone excess, pregnancy, seizure disorder, cardiovascular disease, chronic UTI, renal insufficiency, and following bariatric surgery, to reduce the risk of kidney stone formation.

As used herein, the term “multivitamin” means a dietary supplement containing all or most of the vitamins that may not be readily available in the diet.

The customized multivitamin of the present invention is specifically designed to lower the risk for developing kidney stones while providing the patient with nutrients and compounds important for health and vitality. In a recently completed meta-analysis performed by the present inventors, it has been determined that vitamin C taken as a supplement increases the supersaturation of calcium oxalate in the urine and will lead to the formation of calcium oxalate urinary stones in patients consuming multivitamins containing vitamin C. This risk may be particularly higher in patients with a prior known history of urinary stone formation, or with a family history of stone formation.

By contrast, dietary vitamin C such as is obtained from consuming oranges or orange juice and other citrus fruits has not been definitively linked to urinary stone formation. Most healthy adults can obtain the vitamins and minerals their body needs from the foods they eat. Realizing that sometimes getting your vitamins and minerals from the foods you eat day in and day out can sometimes be challenging, the multivitamin of the present invention is specially tailored to provide enough of the important ingredients, while avoiding excess supplementation of ingredients which can be easily obtained by consuming a broad well balanced diet.

An increase of vitamin C has been shown to be directly linked to an increased risk of kidney stone formation. There is also evidence linking calcium and vitamin D supplementation to an increased risk for calcium-based stone formation, although not as compelling as the evidence linking increased vitamin C supplementation and kidney stone formation. As such, the long-term use of supplements containing large amounts of both calcium and vitamin D can lead to elevated levels of calcium in a patient's blood and subsequently in the urine. This increased calcium in both the blood and urine could increase the risk for formation of calcium based urinary stones. In contrast, dietary consumption of calcium such as including two or more servings of dairy in the diet have been shown to lower the risk of urinary stones presumably by facilitation of the complexing of enteral calcium with enteral oxalate resulting in a stable complex leading to less absorption of calcium oxalate and subsequently a lower risk for calcium oxalate stone formation.

The amount of vitamin C included in the formulation is significantly reduced or completely eliminated from the multivitamin formulations of the present invention. In another embodiment of the present invention, the vitamin C is in the range of from about 20 mg to about 100 mg. In another embodiment of the present invention, the vitamin C is less than about 20 mg. In another embodiment of the present invention, the vitamin C is substantially removed from the multivitamin.

The amount of vitamin D3 included in the formulation is also significantly reduced or completely eliminated from the multivitamin formulations of the present invention. In one embodiment of the present invention, the amount of vitamin D3 is preferably less than about 25 mcg, and more preferably less than about 5 mcg. In another embodiment of the present invention, the vitamin D3 is substantially removed from the multivitamin.

The amount of Calcium included in the formulation is also significantly reduced or completely eliminated from the multivitamin formulations of the present invention. In one embodiment of the present invention, the amount of calcium contained in the multivitamin is preferably less than about 250 mg, more preferably less than about 50 mg, and most preferably less than about 25 mg. In another embodiment of the present invention, calcium is substantially removed from the multivitamin.

The multivitamin formulations of the present invention are specifically formulated to eliminate or significantly reduce all components known to increase the risk for urinary stone formation, and to also include components known to decrease the risk of urinary stone formation such as magnesium, citrate, phytate, and banana stem extract.

Magnesium is known to lower calcium oxalate stone-forming risk through multiple mechanisms. Magnesium binds oxalate in the digestive tract and inhibits the formation of calcium oxalate crystals in urine. Higher magnesium consumption is significantly associated with lower risk of kidney stones.

The most common types of kidney stones, calcium oxalate, and uric acid tend to form in acidic urine. Citrate protects against these types of stones by alkalinizing urine, binding calcium in the gut and in the urine, and inhibiting calcium oxalate crystallization. Therefore, in one embodiment of the present invention, magnesium citrate as a salt is included in a dose less than 1900 mg, preferably between about 200 and about 1000 mg, and most preferably between about 300 and about 500 mg. In another embodiment of the present invention, magnesium citrate as a salt is included in a dose of about 350 mg.

Another compound known to lower the risk for urinary stone formation is phytic acid, or phytate. Phytic acid or phytate, the principal storage form of natural phosphorus in many plant tissues, can form complexes with metals (calcium) and therefore reduce their bioavailability in the gastrointestinal tract. The active ingredient is called inositol hexaphosphate (IP-6), which is known to protect against the development of osteoporosis. Osteoporosis increases the amount of calcium excreted in the blood and urine, increasing the risk of kidney stone formation. Supplementation with IP-6 provides protection against pathological abnormalities in the calcification-decalcification processes. IP-6 acts by preventing crystallization of calcium salts in bodily fluids whether of the oxalate or phosphate variety, thereby inhibiting the crystallization of calcium oxalate and calcium phosphate into kidney stones. It is known that 120 mg per day of IP-6 significantly reduces the formation of calcium oxalate crystals in the urine of people with a history of kidney stone formation. Therefore, in one embodiment of the present invention, IP-6 is included in a dose of about 120 mg/day. In another embodiment of the present invention, IP-6 is preferably included in a dose less than 500 mg, more preferably less than about 200 mg, and most preferably in a dose between about 80 mg and about 160 mg.

It is also known from preliminary rat studies that banana stem extract may decrease the secretion of oxalate glycolic and phosphorus excretion in the urine in hyperoxaluric rats. The present inventors also believe that there are further benefits for calcium oxalate stone formers from the consumption of banana stem extract. Therefore, in one embodiment of the present invention, banana stem extract is optionally included in a dose between about 20 mg and about 200 mg, more preferably between about 20 mg and about 100 mg, and most preferably about 50 mg.

It is also specifically envisioned that multivitamin formulations of the present invention may include additional vitamins and minerals typically found in multivitamin formulations (such as Centrum® Silver, for example), such as vitamins A, E, and B complex.

In order to provide a better understanding of the foregoing, the following non- limiting examples are offered. Although the examples may be directed to specific embodiments, they are not to be viewed as limiting the invention in any specific respect. In particular the dosage of vitamin C, calcium, magnesium citrate, IP-6, and banana stem extract, are merely exemplary, and may be varied as described herein.

In one illustrative embodiment of the present invention, the following multivitamin formulation is provided:

| | |
|---|---|
| Lutein | 10 mg |
| Lycopene | 30 mg |
| Vitamin A | 450 mcg |
| Vitamin D3 | 5 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Calcium (as calcium | 50 mg |
| citrate) | |
| Folic Acid | 400 mcg |
| | (667 mg Folate) |
| Pantothenic Acid | 5 mg |
| Iodine | 150 mcg |
| Zinc | 11 mcg |
| Selenium | 55 mcg |
| Magnesium Citrate | 350 mg |
| | (input amount) |
| Copper | 500 mcg |
| Manganese | 1 mg |
| Insitol hexaphosphate | 120 mg |
| Banana Stem (optional) | 50 mg |

In another illustrative embodiment of the present invention, the following composition for an adult gummy is provided:

| | | |
|---|---|---|
| Vitamin A | 600 mcg | |
| Vitamin C | 30 mg | |
| Vitamin D3(1,000 IU) | 25 mcg | |
| Vitamin E | 18 mg | |
| Riboflavin | 0.26 mg | |
| Niacin | 3.2 mg | |
| Vitamin B6 | 1.6 mg | |
| Folate (24 mcg folic | 40 mcg | DFE = dietary |
| acid) | DFE | folate equivalent |
| Vitamin B12 | 2.4 mcg | |
| Biotin | 30 mcg | |
| Pantothenic Acid | 3 mg | |
| Iodine | 80 mcg | |
| Zinc | 5 mg | |
| Manganese | 0.45 mg | |

In yet another illustrative embodiment of the present invention, the following composition for an adult tablet is provided:

| | |
|---|---|
| Vitamin A (29% as Beta Carotene) | 1,050 mcg |
| Vitamin C | 60 mg |
| Vitamin D3 | 25 mcg |
| | (1,000 IU) |
| Vitamin E | 13.5 mg |
| Vitamin K | 25 mcg |
| Thiamin | 1.5 mg |
| Riboflavin | 1.7 mg |
| Niacin | 20 mg |
| Vitamin B6 | 2 mg |
| Folate (400 mcg folic acid) | 667 mcg |
| Vitamin B12 | 6 mcg |
| Biotin | 30 mcg |
| Pantothenic Acid | 10 mg |
| Calcium | 200 mg |
| Iron | 18 mg |
| Phosphorus | 20 mg |
| Iodine | 150 mcg |
| Magnesium | 50 mg |
| Zinc | 11 mg |
| Selenium | 55 mcg |
| Copper | 0.5 mg |
| Manganese | 2.3 mg |
| Chromium | 35 mcg |
| Molybdenum | 45 mcg |
| Chloride | 72 mg |
| Potassium | 80 mg |

In yet another embodiment of the present invention, a method for customizing a multivitamin for reducing the risk of kidney stone formation in a patient is provided. The method comprises assessing one or more criteria selected from history and physical exam, serum studies, spot urine parameters or 24 hour urine collections. Based on this assessment, a custom multivitamin can be administered having a formulation in accordance with certain teachings of the present invention.

Examples of patient history may include diabetes, quadriplegia, paraplegia, neurogenic bladder, malabsorption. Patients with neurogenic bladder will need supplements designed to prevent infection and to lower calcium and phosphate in the urine. Patients with neurogenic bladder will be directed to the supplement designed for the prevention of infection. Patients with malabsorption will be directed to a low oxalate diet along with a multivitamin with no or low vitamin C.

Examples of serum factors may include but are not limited to uric acid, calcium, pH, Mg, and citrate levels. In patients with high uric acid levels patients will be placed on a supplement with magnesium citrate and potassium citrate. Patients with high serum calcium may be placed on a supplement with low or no calcium. Patients with acid pH may be placed on a low meat diet combined with citrate or other agents to provide alkali. Patients with low citrate and magnesium may be supplemented with these compounds.

In one aspect of the present invention, a 24-hour urine collection such as Litholink, mission, or any other 24 hour urine collection may be utilized. In addition, to improve compliance, a spot urine may be used to determine abnormalities in the urine.

In another embodiment of the present invention, a multivitamin formulation dispenser would be provided, in which a urine sample is submitted to be analyzed and then the dispenser would then dispense the appropriate vitamin formulation to correct the underlying metabolic deficiencies.

In yet another embodiment of the present invention, the patient urinates on a card, and the card based upon the pH, amount of oxalate, calcium, uric acid and presence of infection (nitrite, leukocyte exterase) would specify the appropriate vitamin formulation to correct the underlying metabolic deficiencies.

In order to provide a better understanding of the foregoing, the following non-limiting examples are offered. Although the examples may be directed to specific embodiments, they are not to be viewed as limiting the invention in any specific respect. In particular the dosage of vitamin C, calcium, magnesium citrate, IP-6, and banana stem extract, are merely exemplary, and may be varied as described herein.

In a first illustrative embodiment, for a patient with a stone that is 100% uric acid, then the appropriate vitamin formulation could be:

| | |
|---|---|
| Calcium | 250 mg |
| Vitamin D | 10 mcg |
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |
| Tart cherry extract | 100 mg |
| Potassium Citrate | 283 mg |

In a second illustrative embodiment, for a patient with a pure struvite urinary stone or for patients with recurrent urinary tract infections, then the appropriate vitamin formulation could be:

| | |
|---|---|
| Vitamin C | 100 mg |
| D-Mannose | 900 mg |
| Cranberry juice extract | 800 mg |
| Calcium | 150 mg |
| Vitamin D | 10 mcg |
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |

In a third illustrative embodiment, for a patient with a stone that is 100% calcium oxalate, then the appropriate vitamin formulation could be:

| | |
|---|---|
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |

In a fourth illustrative embodiment, for a patient with a stone that is mixed, but more than 66% of one type, the appropriate vitamin formulation could be the vitamin corresponding to the majority component.

In a fifth illustrative embodiment, if the stone was more than 33% of the two different components calcium oxalate and calcium phosphate, the appropriate vitamin formulation could be:

| | |
|---|---|
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |

In a sixth illustrative embodiment, if the stone was more than 33% of the two different components calcium oxalate and uric acid, the appropriate vitamin formulation could be:

| | |
|---|---|
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |
| Tart cherry extract | 100 mg |

In a seventh illustrative embodiment, if the stone was more than 33% of the two different components calcium phosphate and struvite, the appropriate vitamin formulation could be:

| | |
|---|---|
| D-Mannose | 900 mg |
| Cranberry juice extract | 800 mg |
| Vitamin D | 10 mcg |
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |

In an eighth illustrative embodiment, if the stone was more than 33% of the two different components calcium oxalate and struvite, the appropriate vitamin formulation could be:

| | |
|---|---|
| D-Mannose | 900 mg |
| Cranberry juice extract | 800 mg |
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |

In a ninth illustrative embodiment, if the stone was formed by a patient with cystinuria (cystine stones), the appropriate vitamin formulation could be:

| | |
|---|---|
| Calcium | 250 mg |
| Vitamin D | 10 mcg |
| Lutein | 250 mcg |
| Lycopene | 30 mcg |
| Vitamin A | 450 mcg |
| Vitamin E | 10 mg |
| Thiamin (B1) | 1.5 mg |
| Niacin (B3) | 20 mg |
| Vitamin B 6 | 2 mg |
| Vitamin B 12 | 6 mcg |
| Folic Acid | 400 mcg |
| | (667 mg Folate) |
| Pantothenic Acid | 5 mg |
| Iodine | 150 mcg |
| Zinc | 11 mg |
| Selenium | 55 mcg |
| Magnesium Citrate | 350 mg |
| | (input amount) |
| Copper | 500 mcg |
| Manganese | 1 mg |
| Proprietary Blend | |
| Insitol hexaphosphate | 120 mg |
| Banana Stem (optional) | 50 mg |
| Tart cherry extract | 100 mg |
| Potassium Citrate | 283 mg |

In all of the above formulations, the amounts could vary by plus or minus 10% to allow for degradation over time.

In general a person having skill in the art will appreciate the multivitamin formulations described herein may be administered orally in one or more tablets, capsules, gummies, powdered packet formulation, liquid formulation, or as a drink. For all embodiments described herein, the tablet/capsule size is customizable to the patient's wishes and/or metabolic situation. For example, patients could take a smaller vitamin at three different time periods during the day to spread out the citrate alkalinization to obtain a balanced alkaline urine pH (as is desirable for a patient with uric acid stones). As another example, tablet/capsule size could be customized to patient preference and the size of the patient's esophagus. As a further example, patients could take a smaller vitamin at two to three different time periods during the day such that urinary acidification is spread over the day (as is desirable for struvite stone formers).

In yet another embodiment of the present invention, a method for reducing the risk of kidney stone formation in a patient is provided. The method includes assessing one or more metabolic conditions of the patient, wherein the metabolic conditions are selected from Gout, diabetes, parathyroid hormone excess, pregnancy, seizure disorder, cardiovascular disease, chronic UTI, renal insufficiency, immobility such as seen in quadriplegia, paraplegia or astronauts, and following gastric bypass or bariatric surgery. Based on this assessment, a custom multivitamin can be administered having a formulation in accordance with certain teachings of the present invention.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings therein. It is therefore evident that the particular embodiments disclosed above may be altered or modified and all such variations are considered within the scope and spirit of the present invention.

What is claimed is:

1. A multivitamin formulation for reducing the risk of kidney stone formation, comprising:
less than about 100 mg vitamin C;
less than about 25 mcg vitamin D3;
less than about 250 mg calcium;
between about 200 mg and about 1000 mg magnesium citrate; and
between about 80 mg and about 160 mg inositol hexaphosphate (IP-6).

2. The formulation of claim 1, further comprising between about 20 mg and about 200 mg banana stem extract.

3. The formulation of claim 1, wherein the vitamin C is less than 20 mg.

4. The formulation of claim 1, further comprising substantially no vitamin C.

5. The formulation of claim 1, wherein the vitamin D3 is less than about 5 mcg.

6. The formulation of claim 1, further comprising substantially no vitamin D3.

7. The formulation of claim 1, wherein the calcium is less than about 50 mg.

8. The formulation of claim 1, wherein the calcium is less than about 25 mg.

9. The formulation of claim 1, further comprising substantially no calcium.

10. The formulation of claim 1, wherein the magnesium citrate is between about 300 and about 500 mg.

11. The formulation of claim 1, wherein the IP-6 is between about 100 mg and about 140 mg.

12. The formulation of claim 2, wherein the banana stem extract is between about 20 mg and about 100 mg.

13. The formulation of claim 1, wherein the composition is administered orally in one or more tablets, capsules, gummies, powdered packet formulation, liquid formulation, or as a drink.

14. A multivitamin formulation for reducing the risk of kidney stone formation, comprising:
substantially no vitamin C.

15. The formulation of claim 14, further comprising: substantially no vitamin D3.

16. The formulation of claim 14, further comprising: substantially no calcium.

17. The formulation of claim 14, further comprising magnesium citrate.

18. The formulation of claim 14, further comprising inositol hexaphosphate (IP-6).

19. The formulation of claims 14, further comprising banana stem extract.

20. A multivitamin formulation for reducing the risk of kidney stone formation, comprising:
less than about 100 mg vitamin C.

21. The formulation of claim 20, wherein the vitamin C is less than 20 mg.

22. The formulation of claim 20, further comprising substantially no vitamin C.

23. The formulation of claim 20, further comprising less than about 25 mcg vitamin D3.

24. The formulation of claim 23, wherein the vitamin D3 is less than about 5 mcg.

25. The formulation of claim 23, further comprising substantially no vitamin D3.

26. The formulation of claim 20, further comprising less than about 250 mg calcium.

27. The formulation of claim 26, wherein the calcium is less than about 25 mg.

28. The formulation of claim 26, further comprising substantially no calcium.

29. The formulation of claim 20, further comprising magnesium citrate.

30. The formulation of claim 29, wherein the magnesium citrate is between about 200 and about 1000 mg magnesium citrate.

31. The formulation of claim 29, wherein the magnesium citrate is between about 300 and about 500 mg.

32. The formulation of claim 20, further comprising inositol hexaphosphate (IP-6).

33. The formulation of claim 32, wherein the IP-6 is between about 80 mg and about 160 mg.

34. The formulation of claim 32, wherein the IP-6 is between about 100 mg and about 140 mg.

35. The formulation of claims 20, further comprising banana stem extract.

36. The formulation of claims 35, wherein the banana stem extract is between about 20 mg and about 200 mg.

37. The formulation of claims 35, wherein the banana stem extract is between about 20 mg and about 100 mg.
